SEQUENCE LISTING (1 sequence)

SEQ ID NO: 1 - protein, 4 aa, Homo sapiens
ERRA

(19) United States
(12) Patent Application Publication
(10) Pub. No.: US 2026/0226100 A1
(43) Pub. Date: Aug. 6, 2026

(54) Compound - Diagnostic Marker For Biliary Tract Cancer, Method For Detecting Enzymatic Activity, Method For Diagnosis Of Biliary Tract Cancer, Kit Comprising The Compound, Uses Of The Compound And Method For The Treatment Of Biliary Tract Cancer

(71) Applicant: Urteste S.A., Gdansk (PL)

(72) Inventors: Adam LESNER, Pepowo (PL); Natalia GRUBA, Keblowo (PL)

(73) Assignee: Urteste S.A., Gdansk (PL)

(21) Appl. No.: 19/146,528

(22) PCT Filed: Jan. 8, 2024
(86) PCT No.: PCT/PL2024/050001

§ 371 Date: Jul. 9, 2025

(30) Foreign Application Priority Data
Jan. 9, 2023 (PL) P.443432

(51) Int. Cl. C07K 5/103; G01N 33/542; G01N 33/575

(52) U.S. Cl. CPC C07K 5/1013; G01N 33/542; G01N 33/57557

(57) ABSTRACT

The invention relates to a novel chemical compound—a diagnostic marker—for use in medicine, more specifically in cancer diagnosis, in particular the diagnosis of biliary tract cancer. The invention also relates to an in vitro method for detecting enzymatic activity present in a subject's body fluid, in particular derived from biliary tract cancer cells, using the compound. The invention further relates to an in vitro method for diagnosing biliary tract cancer using the compound, a kit comprising the compound and use of the compound for the detection of enzymatic activity specific to biliary tract cancer and use of the compound for the diagnosis of biliary tract cancer. The invention also relates to the compound for use as a diagnostic marker of biliary tract cancer and a method for the treatment of biliary tract cancer comprising a step of carrying out the method for the diagnosis of biliary tract cancer as defined above using the compound.

The invention relates to a new chemical compound, a diagnostic marker, for use in medicine, more specifically in cancer diagnostics, in particular diagnosis of biliary tract cancer. The invention also relates to an in vitro method for detecting enzymatic activity present in a subject's body fluid, in particular deriving from biliary tract cancer cells, using the compound, an in vitro method for the diagnosis of biliary tract cancer using the compound, a kit comprising the compound, use of the compound for the detection of enzymatic activity specific to biliary tract cancer, use of the compound for the diagnosis of biliary tract cancer, the compound for use as a diagnostic marker for biliary tract cancer. The invention further relates to a method for the treatment of biliary tract cancer comprising a step of carrying out the method for the diagnosis of biliary tract cancer as specified above.

### BACKGROUND OF THE INVENTION

Biliary tract cancer is a relatively rare cancer worldwide. In 2020, 116,000 new cases were reported. The most important etiological factors of the disease that play a role in the pathogenesis of biliary tract cancer include cholelithiasis, chronic inflammation, gallbladder polyps, obesity, toxic exogenous factors, and genetic factors. Biliary tract cancer usually develops asymptomatically and the symptoms of the disease, when present, are uncharacteristic and non-specific. In laboratory tests, elevated serum bilirubin, alkaline phosphatase, gamma-glutamyltranspeptidase and aminotransferase activity are observed. There is also often an elevation of cancer markers in the blood—mainly cancer antigen 19-9 and carcinoembryonic antigen. Biliary tract cancer is classified as a poor prognosis cancer, especially if the diagnosis is made at an advanced stage.

If biliary tract cancer is suspected, a number of imaging tests are indicated. The primary examination, which is very important, inexpensive and safe for the patient, is abdominal ultrasound with assessment of the gallbladder and bile ducts. Complementary tests are abdominal CT scan, MRI, endoscopic retrograde cholangiopancreatography, magnetic resonance cholangiopancreatography. There is currently no test that can be used for the early detection of biliary tract cancer. It is known that the process of initiation, growth and dissemination of cancer cells involves many factors, including many enzymes, in particular hydrolytic enzymes, especially proteolytic enzymes. Such enzymes catalyse enzymatic cleavage (hydrolytic or proteolytic) of proteins and peptides into smaller fragments thereof. This process enables cancer cells to expand by colonizing new tissues, enhancing the process of blood vessels formation (angiogenesis), which enables effective delivery of nutrients to a tumour. Moreover, these enzymes are present as a result of death of healthy cells due to a tumour growth process. All these processes form a characteristic and specific profile of the enzymatic (proteolytic) activity of cancer cells, characteristic to a tumour.

In this field, there are known chromogenic peptide molecules that undergo enzymatic breakdown into smaller fragments resulting in a change or increase in the colour of the solution being tested. This chromogenic effect is a consequence of the release of a chromophore (e.g. 4-nitroanilide or 2-aminobenzoic acid) from a chromogenic peptide molecule.

This type of chromogenic molecules and their uses are known, for example, from the publication by Erlanger B F, Kokowsky N, Cohen W., “The preparation and properties of two new chromogenic substrates of trypsin”, Arch Biochem Biophys., November 1961; 95:271-8 and Hojo K, Maeda M, Iguchi S, Smith T, Okamoto H, Kawasaki K. Amino acids and peptides. XXXV. “Facile preparation of p-nitroanilide analogs by the solid-phase method”, Chem Pharm Bull (Tokyo), November 2000; 48(11):1740-4.

However, the use of this class of compounds in the diagnosis of biliary tract cancer has not been described so far.

Methods for obtaining chromogenic peptides which consist in attaching individual components in appropriate time and stoichiometric conditions are also known in the prior art. The process of attaching consists of subsequent steps in which individual elements (amino acid derivatives) are attached, residues are washed off and protecting groups are sequentially removed and washed again. This cycle is repeated for each amino acid residue. The obtained peptide is separated from resin by a reaction in acidic conditions. Subsequently, the solution is separated from resin in the filtration process and then the peptide is precipitated from the solution by means of a non-polar solvent.

Chromogenic peptide compounds appropriate for a specific and early diagnosis of biliary tract cancer or methods to obtain them are however not known in the prior art.

Therefore, in this field there is an urgent need for “a cancer marker” for biliary tract cancer, which would enable an early, sensitive and specific diagnosis of biliary tract cancer in a non-invasive and reliable manner, and for diagnostic methods and treatment methods using such a diagnostic marker.

The object of the present invention is to provide a novel, specific diagnostic marker for biliary tract cancer and diagnostic methods using such a marker for a non-invasive, quick, sensitive and specific, early detection of biliary tract cancer, which would also be appropriate for screening tests, as well as treatment methods using such a marker.

These objects have been achieved by the inventions defined in the attached patent claims, whereas preferred variants thereof are defined in the dependent claims.

### SUMMARY OF THE INVENTION

The invention provides a compound having formula 1:

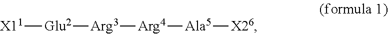

wherein X1 comprises or consists of molecule C1, and X2 comprises or consists of molecule C2,
wherein the pair of molecules C1 and C2 is a pair of florescence donor and fluorescence acceptor,
and wherein the compound undergoes enzymatic cleavage into the fragments X1-Glu-Arg-Arg-Ala-OH (Fragment 1) and X2 (Fragment 2) with a generation of a measurable optical signal upon spatial separation of molecules C1 and C2.

The compound according to the invention preferably undergoes hydrolytic cleavage, more preferably proteolytic.

Preferably, in the compound according to the invention the pair of molecules C1 and C2 is selected from the group consisting of: 2-aminobenzoic acid (ABZ)/5-amino-2-nitrobenzoic (ANB), (ABZ)/pNA, ABZ/ANB-NH₂, ABZ/DNP, ABZ/EDDNP, EDANS/DABCYL, TAM/DANSYL, ABZ/Tyr(3-NO₂), more preferably the pair of C1 and C2 is (ABZ)/pNA or ABZ/ANB-NH₂.

Preferably, the compound according to the invention is a compound having formula 2: ABZ-Glu-Arg-Arg-Ala-ANB-NH₂ (formula 2) or a compound having formula 3: ABZ-Glu-Arg-Arg-Ala-pNA (formula 3).

More preferably, the compound according to the invention undergoes hydrolytic cleavage with the generation of the following fragment 1: ABZ-Glu-Arg-Arg-Ala-OH and fragment 2: ANB-NH₂.

The invention further provides an in vitro method for detecting enzymatic activity present in a subject's body fluid, in particular deriving from biliary tract cancer cells, comprising:
a) contacting a sample of body fluid with the compound having formula 1:

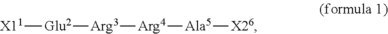

wherein X1 comprises or consists of molecule C1 and X2 comprises or consist of molecule C2,
wherein the pair of molecules C1 and C2 is a pair of a fluorescence donor and a fluorescence acceptor,
and wherein the said compound undergoes enzymatic cleavage into the fragments X1-Glu-Arg-Arg-Ala-OH (fragment 1) and X2 (fragment 2), and
b) detecting a measurable optical signal which is generated upon spatial separation of molecules C1 and C2.

In the method for detecting enzymatic activity according to the invention, enzymatic activity is preferably hydrolytic activity, more preferably proteolytic activity.

In the method for detecting enzymatic activity according to the invention as the said compound the compound having formula 2: ABZ-Glu-Arg-Arg-Ala-ANB-NH₂ (formula 2) or a compound having formula 3: ABZ-Glu-Arg-Arg-Ala-pNA (formula 3) is preferably used.

In the method for detecting enzymatic activity according to the invention as the said body fluid preferably a urine, more preferably human urine, is used.

The invention also relates to an in vitro method for the diagnosis of biliary tract cancer, wherein the presence or absence of biliary tract cancer in a subject is detected by measuring enzymatic activity specific to biliary tract cancer in a body fluid sample from an examined subject, and wherein the absence of the said enzymatic activity indicates the absence of biliary tract cancer, whereas the presence of the said enzymatic activity indicates the presence of biliary tract cancer.

In the method for detecting/diagnosis of biliary tract cancer according to the invention, the detection of enzymatic activity is carried out by the method for detecting enzymatic activity as defined above.

In the method for detecting/diagnosis of biliary tract cancer according to the invention, the measurement of the said enzymatic activity is performed using the compound having formula 1:

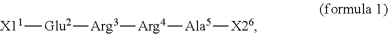

wherein X1 comprises or consists of molecule C1 and X2 comprises or consists of molecule C2,
wherein the pair of molecules C1 and C2 is a pair of a fluorescence donor and a fluorescence acceptor,
and wherein the said compound undergoes enzymatic cleavage into the fragments X1-Glu-Arg-Arg-Ala-OH (fragment 1) and X2 (fragment 2) with the generation of a measurable optical signal upon spatial separation of molecules C1 and C2.

In the method for detecting/diagnosis of biliary tract cancer according to the invention, the said body fluid sample is preferably incubated with the said compound in a measurement buffer having neutral or alkaline pH, more preferably physiological, within the range of sample-to-measurement buffer ratio of from 1:2 to 1:10, preferably 1:5.

In the method for detecting/diagnosis of biliary tract cancer according to the invention, the said compound is preferably used at a concentration of 0.1-10 mg/mL, in particular 0.25-7.5 mg/mL.

In the method for detecting/diagnosis of biliary tract cancer according to the invention, as the said compound the compound having formula 2: ABZ-Glu-Arg-Arg-Ala-ANB-NH₂ (formula 2) or a compound having formula 3: ABZ-Glu-Arg-Arg-Ala-pNA (formula 3) is preferably used.

In the method for detecting/diagnosis of biliary tract cancer according to the invention, as the said sample a urine sample, more preferably human urine, is preferably used.

In the method for detecting/diagnosis of biliary tract cancer according to the invention, the measurement of the said enzymatic activity preferably comprises the measurement of absorbance intensity within the range of 300-500 nm, more preferably 380-430 nm, in particular 405 nm, during 40-60 minutes, at a temperature within the range of 25-40° C., more preferably 36-38° C.

The invention further provides a kit comprising any compound according to the invention as defined above and a measurement buffer.

In the kit according to the invention, the said compound is preferably the compound having formula 2: ABZ-Glu-Arg-Arg-Ala-ANB-NH₂ or a compound having formula 3: ABZ-Glu-Arg-Arg-Ala-pNA.

The invention also provides the use of any compound according to the invention as defined above for the detection of enzymatic activity specific to biliary tract cancer.

The invention also provides the use of any compound according to the invention as defined above for the diagnosis of biliary tract cancer.

Preferably, in such use the diagnosis of biliary tract cancer comprises the detection of primary biliary tract cancer, detection of Minimal Residual Disease after surgical resection of biliary tract cancer and/or detection of biliary tract cancer recurrence.

Preferably, the compound in the uses according to the invention is the compound having formula 2: ABZ-Glu-Arg-Arg-Ala-ANB-NH₂ or a compound having formula 3: ABZ-Glu-Arg-Arg-Ala-pNA.

The invention further provides any of the compounds according to the invention as defined above for use as a diagnostic marker for the detection of biliary tract cancer.

Preferably, the compound for use as the diagnostic marker according to the invention is the compound having formula 2: ABZ-Glu-Arg-Arg-Ala-ANB-NH₂ or a compound having formula 3: ABZ-Glu-Arg-Arg-Ala-pNA.

The invention further provides a method for the treatment of biliary tract cancer, wherein
a) the presence of enzymatic activity specific to biliary tract cancer is detected by any method as defined above in a body fluid sample from an examined subject, and
b) if the presence of the said enzymatic activity is found in the said sample, a treatment for biliary tract cancer is applied in the subject.

Preferably, in the method for the treatment according to the invention, after the end of the treatment in accordance with point b), the said enzymatic activity specific to biliary tract cancer is monitored at predetermined time intervals.

Preferably, in the method for the treatment according to the invention, a urine sample, preferably human urine, is used as the sample.

Preferably, in the method for the treatment according to the invention, the compound having formula 2: ABZ-Glu-Arg-Arg-Ala-ANB-NH₂ or a compound having formula 3: ABZ-Glu-Arg-Arg-Ala-pNA is used as the said compound.

### DETAILED DESCRIPTION OF THE INVENTION

It is to be understood that the present invention is defined in the appended claims. The present description illustrates various, non-limiting embodiments and examples of the invention. The present invention is not limited to any particular methodology, protocol or reagents used to carry it out, unless indicated otherwise. The terms as well as scientific and technical expressions as used herein have meanings commonly known and used by persons skilled in the art of the present invention. For the sake of clarity however, the following expressions/terms and acronyms used in the patent shall be understood as follows:

A chromogenic compound or a chromogenic molecule means a compound having chromogenic properties. Chromogenic properties mean the ability of a compound to form a coloured product.

A fluorescent compound or a fluorescent molecule means a compound having fluorogenic properties. Fluorogenic properties mean the ability of a compound to form a product emitting fluorescence.

NMP stands for N-methylpirrolidone; DMF stands for dimethylformamide; DCM stands for methylene chloride or dichloromethane; pNA stands for 4-nitroaniline or para-nitroaniline; ABZ stands for 2-aminobenzoic acid, ANB-NH₂ stands for amide of 5-amino-2-nitrobenzoic acid; Boc stands for tert-butyloxycarbonyl group; Fmoc stands for 9-fluorenylometoxycarbonyl group; and TFA stands for trifluoroacetic acid.

In the context of the present invention, the term diagnosis of biliary tract cancer shall be understood to mean identification of this disease, in particular at its early stage at which other diagnostic methods are not sensitive and/or specific enough. As used herein, the diagnosis of biliary tract cancer also comprises the detection of Minimal Residual Disease (MRD) after surgical resection of biliary tract cancer and detection of biliary tract cancer recurrence after previously completed treatment of biliary tract cancer.

In the context of the present invention, the term: treatment of biliary tract cancer shall be understood to mean a treatment at an early stage of progression of the disease, which makes it possible to significantly prolong survival time and improve the quality of life of the diseased subjects.

In the context of the present invention, the term: monitoring shall be understood to mean the diagnosing of Minimal Residual Disease (MRD))—the presence of a small number of cancer cells that have survived in the organism (during treatment or remission), in the amounts undetectable by means of standard diagnostic methods.

In the context of the present invention, the term: subject shall be understood to mean a human subject or a mammal that is suspected to have biliary tract cancer, or alternatively a human subject or a mammal belonging to a group with an increased risk of biliary tract cancer, or a human subject or a mammal after resection of biliary tract cancer or after a finished treatment of biliary tract cancer. The subject is preferably a human subject.

The compounds according to the invention have chromogenic properties due to the presence of a chromophore, and fluorogenic properties, i.e. they contain molecules of a fluorescence donor and acceptor. Due to their structure, developed in such a way that an increase in colour is observed in the wavelength range of 380-440 nm, specifically as a result of contact of a tested body fluid sample from an examined subject with biliary tract cancer, whereas such an effect is not observed in the reaction with a body fluid sample from a healthy subject or a subject with the diagnosis of another type of cancer, these compounds make it possible to detect enzymatic activity specific to biliary tract cancer, and in particular to diagnose biliary tract cancer in a specific and sensitive manner, also at an early stage of progression of this cancer. The examined subject is preferably a human subject. The body fluid is preferably urine, more preferably human urine.

In the first aspect of the present invention, a novel chemical compound is provided which compound has formula 1:

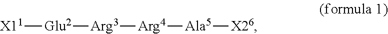

wherein X1 is an amino acid derivative or a peptide fragment comprising molecule C1, or X1 consists of such a molecule C1, and X2 is an amino acid derivative or a peptide fragment comprising molecule C2, or X2 consists of such a molecule C2, wherein the pair of molecules C1 and C2 is a pair of a fluorescence donor and a fluorescence acceptor. The superscripts indicate subsequent positions of residues in the compound according to the invention and the sequence of attaching of the residues during synthesis. In accordance with the present invention, in this context chemical formula 1 can be alternatively written without indicating the numbering of residues. The core of all compounds according to the invention is tetrapeptide with the indicated sequence of 4 amino acids, i.e. Glu-Arg-Arg-Ala (the notation in the three-letter amino acid abbreviation format equivalent to the notation: DTFI in the one-letter amino acid abbreviation format), which sequence is also presented in the Sequence Listing as sequence no. 1.

The compound according to the invention undergoes enzymatic cleavage into the fragments: X1-Glu-Arg-Arg-Ala-OH (fragment 1) and X2 (fragment 2) with the generation of a measurable optical signal upon spatial separation of molecules C1 and C2. The measurable optical signal is measured by a method for measuring a change in absorbance/fluorescence after the enzymatic cleavage of the compound. Preferably, molecules C1 and C2 are separated from each other by not more than 10 amino acid residues, which ensure efficient quenching of the fluorescence donor by the fluorescence acceptor. It is obvious for the skilled person that the key factor is the distance between the fluorescence donor and acceptor. Therefore, where the amino acid sequence separating molecules C1 and C2 is folded into a twisted or condensed secondary structure, resulting in a proximity of molecules C1 and C2 relative to the primary structure, the distance between molecules C1 and C2 can be greater than 10 amino acid residues.

The compound, due to its chromogenic properties and the presence of a reactive site at the position 5 enabling enzymatic (preferably proteolytic) cleavage into smaller fragments, is particularly suitable for use as a diagnostic marker, in particular a specific diagnostic biomarker for biliary tract cancer, in particular for use in the early diagnosis of biliary tract cancer.

In preferred embodiments, the compound according to the invention undergoes hydrolytic cleavage, more preferably proteolytic cleavage.

In preferred embodiments the pair of molecules C1 and C2 is selected from a group consisting of: 2-aminobenzoic acid (ABZ)/5-amino-2-nitrobenzoic acid (ANB), (ABZ)/pNA, ABZ/ANB-NH₂, ABZ/DNP, ABZ/EDDNP, EDANS/DABCYL, TAM/DANSYL, ABZ/Tyr(3-NO₂), more preferably the pair of molecules C1 and C2 is ABZ/pNA or ABZ/ANB-NH₂.

In preferred embodiments, the compound according to the invention is:
the compound having formula 2:

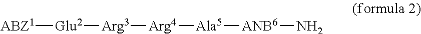

the compound having formula 3:

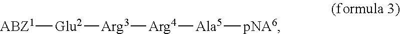

wherein ABZ stands for 2-aminobenzoic acid, ANB-NH₂ stands for amide of 5-amino-2-nitrobenzoic acid, and pNA stands for 4-nitroaniline.

The compound is subject to hydrolytic cleavage with the generation of the following fragment 1: ABZ-Glu-Arg-Arg-Ala-OH and fragment 2: ANB-NH₂ in the case of the compound having formula 2, whereas in the case of the compound having formula 3, with the generation of the following fragment 1: ABZ-Glu-Arg-Arg-Ala-OH and fragment 2: pNA. Thus, fragments 2 are free chromophores.

Spatial separation of molecules C1 and C2 being a result of the enzymatic cleavage of the compound according to the invention causes generation of a measurable optical signal because fluorescence emitted from the fluorescence donor is no longer quenched by the fluorescence acceptor. Such measurable optical signal can be detected preferably at a wavelength of 300-500 nm, more preferably 380-430 nm.

The compounds according to the invention can be obtained by known methods. For example, they can be obtained using a method for obtaining chromogenic peptides which consists in carrying out the process on a solid support in the form of a resin having an Fmoc group, which is removed in the course of the reaction. For example, it can be an amide resin, e.g. Teenage S RAM or RinkAmide, but any other commercially available resin can also be used. The resin used to carry out the process should be properly prepared. The preparation of the resin consists in increasing its volume by repeated washing with hydrophobic solvents. Preferably, a resin with a deposition of 0.23 mmol/g is used. The Fmoc protecting group must be removed from the resin by washing it with a 20% solvent solution.

Then, the known processes for obtaining chromogenic peptides comprise attaching individual components in appropriate time and stoichiometric conditions. The attaching process consists of subsequent steps in which individual elements (amino acid derivatives) are attached, residuals are washed off and protecting groups are successively removed and washed again. This cycle is repeated for each amino acid residue. The obtained peptide is separated from the resin by a reaction under acidic conditions. Then, the solution is separated from the resin in the filtration process and then the peptide is precipitated from the obtained solution by means of a non-polar solvent. The peptide precipitate obtained in this way is centrifuged.

Exemplary, detailed but non-limiting, method for the synthesis of the compounds according to the invention is described below and in Example 1 below.

The method for the synthesis of the compound according to the invention consists in that the process is carried out on a solid support in the form of a resin, preferably having an Fmoc group, wherein before the start of the process the solid support is prepared by increasing its volume by repeated washing with hydrophobic solvents, preferably dimethylformamide, methylene chloride or N-methylpyrrolidone, and removing the Fmoc protecting group, preferably by washing with 10-30% piperidine solution, in solvents such as dimethylformamide, methylene chloride or N-methylpyrrolidone.

Then, the method is carried out in subsequent steps:
a) Deposition of 5-amino-2-nitrobenzoic acid, ANB (or another chromophore suitable for use according to the invention as defined in the claims) on the resin is preceded by washing the solid support with a 3-6% solution of N-methylmorpholine (NMM) in DMF, and then DMF, after which a solution of ANB in DMF is prepared to which TBTU, DMAP and finally diisopropylethylamine (DIPEA) are successively added in the following excess relative to the polymer deposition: ANB/TBTU/DMAP/DIPEA, 3:3:2:6; the mixture prepared in this way is added to the resin and is mixed until homogenous, after which the resin is filtered off under reduced pressure and is washed with solvents such as DMF, DCM and isopropanol, and then the attaching of ANB to the resin is continued using hexafluorophosphate-O-(7-azabenzotriazol-1-yl)-N,N,N,N′,N′-tetramethyluronium (HATU), and then hexafluorophosphate-O-(benzotriazol-1-yl)-N,N,N′,N′-tetramethyluronium (HBTU) in excess, and after finishing, the solid support is washed successively with DMF, DCM and isopropanol, and is gently dried;
b) Attachment of the amino acid residue to ANB is carried out by a reaction with the amino acid derivative—Fmoc-Ala-OH, wherein at least fivefold molar excess of the amino acid derivative relative to the resin is dissolved in anhydrous pyridine and is contacted with the resin with deposited ANB, after which the whole is cooled to a temperature not lower than −20° C., and then POCl₃ is added in the ratio 1:1 relative to the amount of the amino acid derivative used and the whole is mixed, after which the mixing process is carried out at room temperature and then at an elevated temperature and when the reaction is completed, the resin in filtered off under reduced pressure, washed with DMF and MeOH and gently dried, after which the obtained intermediate compound is subjected to the process of acylation, successively attaching a fragment of Glu-Arg-Arg;
c) Acylation of the obtained intermediate compound is carried out using an amino acid derivative, preferably Fmoc-Arg(Pbf)-OH, and subsequently Fmoc-Arg(Pbf)-OH, then Fmoc-Glu(tBu-OH and, at the final step of the synthesis, Boc-Abz-OH, the acylation being carried out in steps from residue 6 to 1, using diisopropylcarbodiimide as a coupling agent, which is used in excess, and after finishing each step the resin is washed with DMF and preferably is subjected to the chloranil test (a test for the presence of free amino groups) in which the attachment of the amino acid derivative is monitored;
d) Removal of the Fmoc protecting group is carried out by washing with 10-30% piperidine solution in DMF and subsequent washings with each of the solvents: DMF, isopropanol and methylene chloride;
e) Separation of the peptide from the resin is carried out using the mixture: TFA:phenol:water:TIPS while maintaining the ratios of 88:5:5:2 v/v/v/v, respectively, the mixture is stirred for the period of at least one hour, preferably three hours, and the obtained precipitate filtered off under reduced pressure, after which it is washed with diethyl ether and the obtained peptide is centrifuged;
f) Preparation of the finished product is carried out by dissolving the peptide in water by means of ultrasound and then it is subjected to lyophilisation.

The second aspect of the present invention provides an in vitro method for detecting enzymatic, preferably proteolytic, activity, present in a subject's body fluid, in particular deriving from biliary tract cancer cells, the method comprising a) contacting the body fluid sample with the compound according to the invention and b) detecting a measurable optical signal which is generated upon spatial separation of molecules C1 and C2 present in the compound according to the invention. In a preferred embodiment of this aspect, the examined subject in this case is a human subject. In another preferred embodiment of this aspect, the body fluid is urine, in particular human urine.

In the preferred embodiment of this aspect, the compound having formula 2: ABZ-Glu-Arg-Arg-Ala-ANB-NH₂ or a compound having formula 3: ABZ-Glu-Arg-Arg-Ala-pNA is used.

The third aspect of the present invention provides an in vitro method for the diagnosis of biliary tract cancer in which the presence or absence or biliary tract cancer in a subject is detected by measuring enzymatic activity specific to biliary tract cancer in a body fluid sample from the examined subject, wherein the absence of the said enzymatic activity indicates the absence of biliary tract cancer whereas the presence of the said enzymatic activity indicates the presence of biliary tract cancer. Such detection of enzymatic activity is preferably carried out using methods for detecting enzymatic activity as discussed above. In the preferred embodiment of this aspect the subject is a human subject. In the preferred embodiment of this aspect the body fluid is urine, in particular human urine. In the preferred embodiment of this aspect the enzymatic activity specific to biliary tract cancer is proteolytic activity. In the preferred embodiment of this aspect the compound having formula 2: ABZ-Glu-Arg-Arg-Ala-ANB-NH₂ or a compound having formula 3: ABZ-Glu-Arg-Arg-Ala-pNA is used.

Moreover, in the preferred embodiment of this aspect, the measurement of the said enzymatic activity in the methods according to the invention comprises the measurement of absorbance intensity within the range of 300-500 nm, preferably 380-430 nm, in particular 405 nm, during 40-60 minutes, at a temperature within the range of 25-40° C., preferably 36-38° C. This enables obtaining a maximally intensive measurable optical signal resulting from an increase in absorbance or fluorescence.

Furthermore, in the preferred embodiments of the said methods according to the invention the measurement of the said enzymatic activity is performed using the compound according to the invention in the range of concentrations of 0.1-10 mg/mL, more preferably at the concentration of 1 mg/mL. In the preferred embodiments of the said methods according to the invention the tested sample is incubated with the compound according to the invention in a measurement buffer having a neutral or alkaline pH, preferably physiological, with a body fluid sample, preferably human urine, with the sample (e.g. of urine) to measurement buffer ratio ranging from 1:2 to 1:10, preferably 1:5. The sample is preferably taken from a subject with a referral for the diagnosis of biliary tract cancer. Preferably, absorbance intensity is measured within the range of 300-500 nm, preferably 380-430 nm, in particular 405 nm, during 40-60 minutes, at a temperature within the range of 25-40° C., preferably 36-38° C. In the aforementioned conditions, a maximally intensive measurable optical signal is obtained resulting from an increase in absorbance or fluorescence.

In the fourth aspect, the present invention provides a kit comprising any compound according to the invention and a measurement buffer. Measurement buffers are known in this art and a buffer suitable for use in the kit according to the invention is, for example, but without limitation, the Tris-HCl buffer. In the preferred embodiment, in the kit according to the invention the said compound is the compound having formula 2: ABZ-Glu-Arg-Arg-Ala-ANB-NH₂ or a compound having formula 3: ABZ-Glu-Arg-Arg-Ala-pNA.

In the fifth aspect, the present invention provides use of the compound according to the invention for the detection of enzymatic activity specific to biliary tract cancer. In the sixth aspect, the present invention provides use of the compound according to the invention for the diagnosis of biliary tract cancer. Preferably, the diagnosis of biliary tract cancer comprises, according to the invention, the detection of primary biliary tract cancer, detection of Minimal Residual Disease after surgical resection of biliary tract cancer and/or detection of biliary tract cancer recurrence after previously completed biliary tract cancer treatment.

In the seventh aspect, the present invention provides the compound according to the invention for use as a diagnostic marker for the detection of biliary tract cancer. In the preferred embodiments of this aspect the said compound is the compound having formula 2: ABZ-Glu-Arg-Arg-Ala-ANB-NH₂ or a compound having formula 3: ABZ-Glu-Arg-Arg-Ala-pNA.

In the eight aspect, the present invention provides a method for the treatment of biliary tract cancer wherein
a) the presence of enzymatic activity specific to biliary tract cancer is detected by any method according to the invention as defined above, in a body fluid sample from the examined subject, and
b) when the said enzymatic activity is found to be present in the said sample, a treatment of biliary tract cancer is applied in the subject.

In the preferred embodiment of the method for the treatment, after completing the treatment according to point b), the said enzymatic activity specific to biliary tract cancer is monitored at predetermined time intervals as known in the art, e.g. every week, every several weeks, every month, every several months, every year or at any other intervals considered to be appropriate by the skilled person, in order to detect Minimal Residual Disease after surgical resection of biliary tract cancer or recurrence. Furthermore, in the preferred embodiment of the method, a urine sample, preferably human urine, is used as the test sample. In the preferred embodiment of the method for the treatment the compound having formula 2: ABZ-Glu-Arg-Arg-Ala-ANB-NH₂ (formula 2) or a compound having formula 3: ABZ-Glu-Arg-Arg-Ala-pNA (formula 3) is used as the said compound.

The advantages of the present invention consist in providing a novel chemical compound having properties that make it suitable for use for specific and sensitive detection of enzymatic activity specific to biliary tract cancer, for use as a diagnostic biomarker for the detection of biliary tract cancer, for use in a fast, non-invasive diagnosis of biliary tract cancer, while enabling the detection of biliary tract cancer at an early stage of its progression. Another advantage is that the diagnostic methods according to the invention can be successfully used in screening tests. This enables full diagnosis at an early stage of cancer progression and consequently a more effective treatment. Early diagnosis enables surgical treatment, which significantly prolongs patient's survival time. It is also important when monitoring the effectiveness of the applied surgical treatment and/or chemotherapy of biliary tract cancer since it is possible to detect Minimal Residual Disease or recurrence, if any.

The present invention will now be illustrated in the following figures and the following examples, which however are not intended to limit, in any manner, the scope of the invention as defined in the claims.

### BRIEF DESCRIPTION OF THE FIGURES

FIG. 1 shows the results of chromatographic analysis of the substrate cleavage, i.e. ABZ-Glu-Arg-Arg-Ala-ANB-NH₂, in a sample of urine from a subject with biliary tract cancer.

FIG. 2 shows the rate of hydrolysis of the substrate—ABZ-Glu-Arg-Arg-Ala-ANB-NH₂— in the samples of urine from subjects with diagnosed biliary tract cancer (samples 1-13) and urine taken from healthy subjects (samples 14-23). Arabic numerals indicate the number of the selected urine sample.

FIG. 3 shows the selectivity of hydrolysis of the substrate—ABZ¹-Glu²-Arg³-Arg⁴-Ala⁵-NH₂ (i.e. compound of formula 2) in urine samples taken from subjects with diagnosed biliary tract cancer and urine samples taken from subjects with the diagnosis of another neoplastic disease. The samples tested for each type of cancer were derived from 20 different patients for each of the tested cancers. The results are mean values for given cancer types. The results show selectivity of substrate cleavage in the case of urine from patients with biliary tract cancer as compared to urine samples from patients suffering from other neoplasms.

FIG. 4 shows the dependence of the hydrolysis level of the substrate—ABZ¹-Glu²-Arg³-Arg⁴-Ala⁵-NH₂, on pH conditions.

### EXAMPLES

The invention is illustrated by the following non-limiting examples. Unless indicated otherwise, the examples below use known and/or commercially available devices, methods, reaction conditions, reactants and sets, which are commonly used in the field to which the present invention belongs and which are recommended by the producers of respective reactants and kits.

### Example 1: Synthesis of the Compound According to the Invention

This example presents the synthesis of one representative compound according to the present invention, namely the compound: ABZ¹-Glu²-Arg³-Arg⁴-Ala⁵-NH₂. The remaining peptides according to the invention can be synthesized in an analogous way. The superscripts indicate subsequent positions of residues in the compound according to the invention and the sequence of attachment of the residues during synthesis. The compounds according to the invention can be alternatively represented by an analogous formula without the indication of residue positions, which does not change the sequence of residues in the compounds according to the invention, as it remains unchanged.

### 1. Obtaining a Chromogenic Peptide

a) The first step of the synthesis was to obtain the chromogenic peptide, which was obtained by solid phase synthesis, on a solid support, using Fmoc/tBu chemistry, i.e. with the use of protection.

A compound having the sequence ABZ¹-Glu²-Arg³-Arg⁴-Ala⁵-NH₂, wherein ABZ is 2-aminobenzoic acid and ANB-NH₂ is amide of 5-amino-2-benzoic acid and ANB is 5-amino-2-benzoic acid, was obtained in the process of solid phase chemical synthesis using the following amino acid derivatives:
Boc-ABZ, Fmoc-Glu(OtBu), Fmoc-Arg(Pbf), Fmoc-Arg(Pbf), Fmoc-Ala.

The synthesis of the compound according to the invention, which can be used as a diagnostic marker for the detection of biliary tract cancer, was carried out on a solid support enabling the conversion of 5-amino-2-beznoic acid into ANB-NH₂ amide, namely amide resin TentaGel S RAM from RAPP Polymere (Germany), with a deposition of 0.23 mmol/g. However, it is possible to use any other amide resin, e.g. Rink Amide (Germany).

The synthesis of the compound was carried out manually using a laboratory shaker. In most of the steps a 25 mL sintered syringe for solid phase synthesis was used as a reactor.

All the obtained final compounds contained 2-aminobenzoic acid (ABZ) at the position 1 of their sequence, i.e. at the N-terminus, and a 5-amino-2-nitrobenzoic acid (ANB) molecule at the position 6, i.e. at the C-terminus. ABZ acts here as a fluorescence donor, while ANB-5-amino-2-nitrobenzoic acid—acts as a fluorescence quencher and simultaneously a chromophore. The peptides contained at least and preferably one reactive site in their sequence, located between the amino acid residue Ala-ANB-NH₂, i.e. at the position 5 of the compound. The synthesis consisting in attaching amino acid derivatives is carried out from residue 6 to 1, i.e. from the C- to N-terminus.

### b) Deposition of ANB on TentaGel S RAM Resin:

The synthesis of peptides was performed on TentaGel S RAM resin from Rapp Polymere with a deposition of 0.23 mmol/g. In the first step, the resin was prepared, including loosening it by the wash cycle. Subsequently, the protection of the Fmoc amino group was removed from the solid support with the 20% solution of piperidine in NMP. Then, the solvent washing cycle was carried out. In order to confirm the presence of free amino groups, a chloranil test was performed.

### Solvent Wash Cycle:

DMF 1×10 minutes; IsOH 1×10 minutes; DCM 1×10 minutes.

### Removal of Fmoc Protection:

DMF 1×5 minutes; 20% piperidine in NMP 1×3 minutes; 20% piperidine in NMP 1×8 minutes.

### Solvent Wash Cycle:

DMF 3×2 minutes; IsOH 3×2 minutes; DCM 3×2 minutes.

### c) Chloranil Test:

The chloranil test consisted in transferring, by means of a spatula, several grains of resin from the reactor—a syringe, into a glass ampule, to which subsequently 100 μL of saturated solution of p-chloranil in toluene and 50 μL of fresh acetaldehyde were added. After 10 minutes, the control of grains colour was carried out.

At that stage, after performing the test, a green colour of the grains was obtained, which evidenced the presence of free amino groups. After confirming the removal of 9-fluorenylmethoxycarbonyl protection from the resin, it was possible to proceed to the next step, the attachment of the ANB derivative (5-amino-2-nitrobenzoic acid).

d) Deposition of 5-amino-2-nitrobenzoic acid on Solid Support:

The first step in the synthesis of the peptide was deposition of ANB on 1 g of resin. Before attaching the chromophore, the resin used for the reaction was washed with the following solvents: DMF, DCM and again DMF, after which the Fmoc-protection was removed from the functional group of the solid support. One cycle of removing the Fmoc-protection comprised the following steps:

### Removal of Fmoc-Protection:

20% piperidine in NMP 1×3 minutes; 20% piperidine in NMP 1×8 minutes.

### e) Washing:

DMF 3×2 minutes; IsOH 3×2 minutes; DCM 3×2 minutes.

### f) Chloranil Test:

The resin with a free amino group was washed with 5% solution of N-methylmorpholine (NMM) in DMF, and then DMF. The procedure of removing the Fmoc-protection and the wash cycle were carried out in a Merrifield vessel. In a separate flask, ANB was dissolved in DMF, and TBTU, DMAP and finally diisopropylethylamine (DIPEA) were subsequently added in the following excess relative to polymer deposition: ANB/TBTU/DMAP/DIPEA, 3:3:2:6 v/v/v/v. The mixture prepared in this way was added to the resin and was stirred for 3 hours. The resin was filtered off under reduced pressure, washed with DMF, DCM and isopropanol, and the entire acylation procedure was repeated twice. To carry out subsequent reactions of attaching ANB to the resin, hexafluorophosphate-O-(7-azabenzotriazol-1-yl)-N,N,N′,N′-tetramethyluronium (HATU) and then hexafluorophosphate-O-(benzotriazol-1-yl)-N,N,N′,N′-tetramethyluronium (HATU) were used. In the last step, the resin was washed successively with DMF, DCM and isopropanol, and was air dried.

### g) Attachment of the C-Terminal Amino Acid Residue (Fmoc-Ala-OH) to ANB:

The corresponding amino acid derivative (9-fold molar excess relative to resin deposition) was dissolved in pyridine and was transferred to the flask containing the resin with deposited ANB. The whole was cooled until the temperature of −15° C. was reached (ice bath: 1 part by weight of NH₄Cl, 1 part by weight of NaNO₃, 1 part by weight of ice). After the desired temperate was reached, POCl₃ was added (in 1:1 ratio to the amount of amino acid derivative used) and the whole was stirred on a magnetic stirrer: for 20 minutes at −15° C., 30 minutes at room temperature and 6 hours at 40° C. (oil bath). When the reaction was completed, the resin was filtered off under reduced pressure, washed with DMF and MeOH and left to dry. In the next stage, the residue was attached in P2 position (Fmoc-Arg(Pbf)).

Every attachment of amino acid residue was preceded by washing the resin with DMF for 5 minutes. Diisopropylcarbodiimide was used as a coupling agent in subsequent attachments. The procedure was repeated twice.

After each acylation, a resin wash cycle was started and then the chloranil test was performed in order to monitor the attachment of the amino acid derivative to free amino acid groups of the resin.

### Solvent Wash Cycle:

DMF 3×2 minutes; IsOH 3×2 minutes; DCM 3×2 minutes.

### Chloranil Test:

As a result of the performed tests, after the first two coupling procedures, the colour of the grains was first green and then grey, so it was necessary to perform another acylation, as a result of which the resin grains tested by the chloranil test were colourless. This evidenced the attachment of ANB to the TentaGel S RAM resin, and thus it was possible to proceed to the next step of peptide synthesis.

### h) Attachment of Subsequent Protected Amino Acid Residues:

The resin together with the attached fragment ANB-Ala, located in the reactor, was washed with DMF, which was followed by deprotection of the Fmoc from the amino group in order to attach the protected amino acid derivative Arg.

### Removal of Fmoc Protection:

DMF 1×5 minutes; 20% piperidine in NMP 1×3 minutes; 20% piperidine in NMP 1×8 minutes.

### Solvent Wash Cycle:

DMF 3×2 minutes; IsOH 3×2 minutes; DCM 3×2 minutes.

### Chloranil Test:

The chloranil test produced a positive result, which was evidenced by the green colour of the resin grains. Thus, it was possible to proceed to the next step—attachment of the amino acid residue Fmoc-Arg(Pbf)-OH.

### Attachment of the Amino Acid Derivative

The process of coupling was preceded by washing the resin with DMF. The composition of the coupling mixture remained unchanged when attaching the protected serine residue.

After the end of each acylation, a solvent wash cycle was performed according to the specified procedure and then the chloranil test for the presence of free amino acid groups in the solution was performed.

### Solvent Wash Cycle

DMF 3×2 minutes; IsOH 3×2 minutes; DCM 3×2 minutes.

### Chloranil Test:

During the test performed after the second acylation the resin grains were colourless, and thus it was possible to proceed to the next step of the synthesis i.e. the introduction of another protected amino acid derivative—Fmoc-Glu(OtBu) and 2-aminobenzoic acid molecule. The coupling processes were performed according to the procedure discussed earlier.

Tests carried out after attaching the above-mentioned residues showed positive results—the resin grains were colourless.

2. Removal of the Peptide from the Solid Support

After the synthesis, the amide of ABZ-Glu-Arg-Arg-Ala-ANB-NH₂ peptide was removed from the solid support and simultaneously the side protection was removed using the mixture: TFA:phenol:water:TIPS (88:5:5:2, v/v/v/v) in a round-bottom flask on a magnetic stirrer.

After 3 hours, the content of the flask was filtered off under reduced pressure in sintered (Schott) funnels and washed with diethyl ether. The obtained sediment was centrifuged on a SIGMA 2K30 centrifuge (Laboratory Centrifuges) for 20 minutes. The precipitate obtained after centrifugation was dissolved in water by means of ultrasound and then it was subjected to lyophilisation. The remaining compounds according to the invention can be obtained in an analogous way.

The identity/characteristics of the novel compound according to the invention were confirmed using the HPLC analysis. The conditions of the HPLC analysis were as follows: RP Bio Wide Pore Supelco C8 column, 250 mm 4 mm, a phase system A 0.1% TFA in water, B: 80% acetonitrile in A), flow rate 1 mL/min, UV detection at 226 nm.

The analysis carried out confirmed that the compound according to the invention was obtained.

### Example 2: Testing the Properties of the Compound According to the Invention as a Cancer Marker

The activity of the novel compounds according to the invention was studied in a group of 20 subjects diagnosed with biliary tract cancer using the representative compound according to the invention. The mechanism of action of the compounds according to the invention, including the representative compound having formula 2, consists in specific enzymatic cleavage, more specifically enzymatic hydrolysis, at the position that leads to the release of free molecules of respective chromophores: ANB-NH₂ (amide of 5-amino-2-nitrobenzoic acid) in the case of the compound having formula 2 or pNA (para-nitroaniline) in the case of the compound having formula 3, which exhibit absorbance at a wavelength of 320-480 nm, especially 380-430 nm, in particular 405 nm. The remaining compounds according to the invention are characterized by the analogous mechanism of action. For this purpose, the representative compound according to the invention, ABZ¹-Glu²-Arg³-Arg⁴-Ala⁵-NH₂, was dissolved in dimethyl sulfoxide (at a concentration of 0.5 mg/mL) and then 50 L of the solution was mixed with 120 L of a buffer (200 mM Tris-HCl, pH 8.0) and 80 L of urine from a subject suffering from biliary tract cancer. The measurement was performed on a 96-well plate designed for measuring absorbance and each sample was analysed in triplicate at the temperature of 37° C. The duration of the measurement was 60 minutes. During the measurement, the wavelength characteristic for the chromophore (ANB-NH₂) being released was monitored at the wavelength 405 nm (range 380-430 nm).

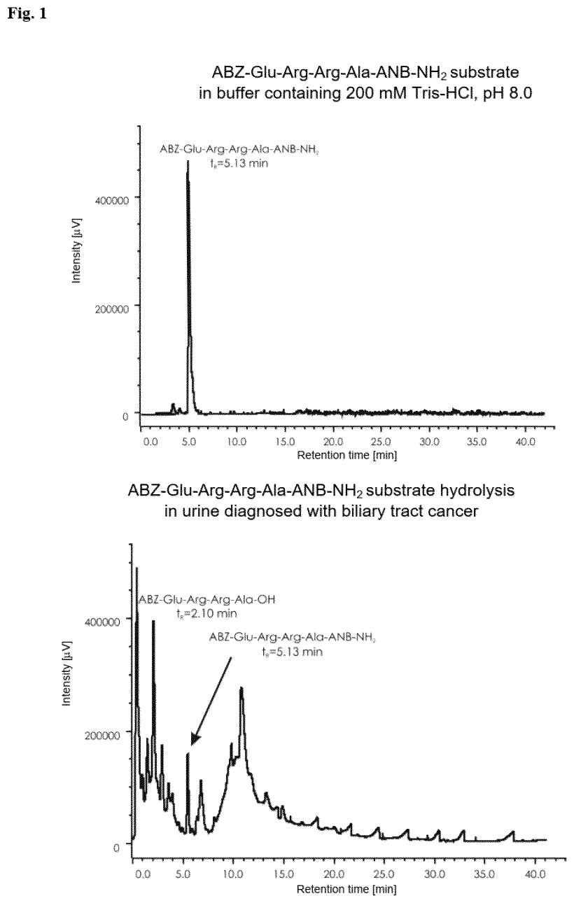

A shown in FIG. 1, the RP HPLC analysis of a randomly selected system comprising urine taken from a person diagnosed with biliary tract cancer indicates that the compound according to the invention cleaves into the peptide fragment ABZ-Glu-Arg-Arg-Ala-OH and the chromophore group of the compound (ANB-NH₂).

The measurement showed that the colour intensity of the solution increased with time in all urine samples taken from persons diagnosed with biliary tract cancer. The observed magnitude of increase in absorbance in time is different for each of the examined samples. A different effect was obtained for 10 samples from healthy subjects since no increase in absorbance within the diagnostic range was observed in any of the 10 tested urine samples.

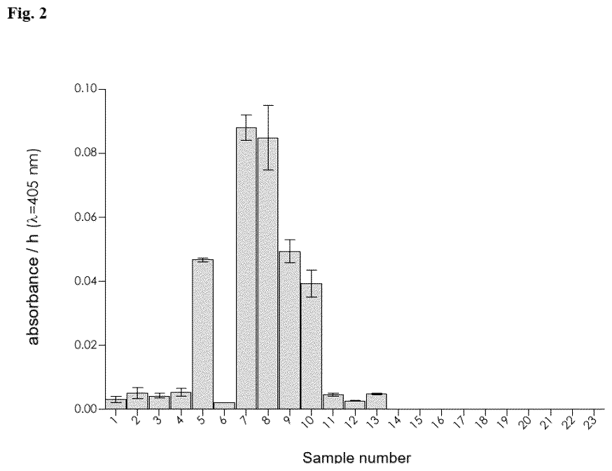

The tests carried out show that all samples 1-13 from persons suffering from biliary tract cancer underwent cleavage, but in the case of samples 1, 6 and 12, cleavage of the substrate, i.e. ABZ-Glu-Arg-Arg-Ala-ANB-NH₂, proceeded less efficiently than in the case of samples 7 or 8 (Table 1 FIG. 2). Such a result may be due to the difference in the activity as well as the amount of enzymes responsible for the enzymatic cleavage (proteolysis). Furthermore, the results presented in Table 1 below indicate that incubating the substrate solution—the compound according to the invention—with urine samples taken from healthy persons (without a cancer diagnosis, marked with Arabic numerals sequentially from 14 to 23) does not lead to an increase in absorbance and thus hydrolysis of the tested compound does not take place. The result indicates the absence of proteolytic enzymes specific/characteristic of biliary tract cancer.

TABLE 1
Results of absorbance analysis

| | | | |
|---|---|---|---|
| 1 | 0.004 | 0.002 | 0.003 |
| 2 | 0.0068 | 0.0034 | 0.0053 |
| 3 | 0.0035 | 0.005 | 0.0048 |
| 4 | 0.004 | 0.0067 | 0.0051 |
| 5 | 0.0474 | 0.0461 | 0.0042 |
| 6 | 0.002 | 0.0021 | 0.0026 |
| 7 | 0.084 | 0.092 | 0.087 |
| 8 | 0.0746 | 0.0948 | 0.084 |
| 9 | 0.053 | 0.0458 | 0.0497 |
| 10 | 0.0436 | 0.035 | 0.041 |
| 11 | 0.0051 | 0.0041 | 0.0048 |
| 12 | 0.0025 | 0.0028 | 0.0032 |
| 13 | 0.0046 | 0.005 | 0.0047 |
| 14 | 0.000000 | 0.000000 | 0.000000 |
| 15 | 0.000000 | 0.000000 | 0.000000 |
| 16 | 0.000000 | 0.000000 | 0.000000 |
| 17 | 0.000000 | 0.000000 | 0.000000 |
| 18 | 0.000000 | 0.000000 | 0.000000 |
| 19 | 0.000000 | 0.000000 | 0.000000 |
| 20 | 0.000000 | 0.000000 | 0.000000 |
| 21 | 0.000000 | 0.000000 | 0.000000 |
| 22 | 0.000000 | 0.000000 | 0.000000 |
| 23 | 0.000000 | 0.000000 | 0.000000 |

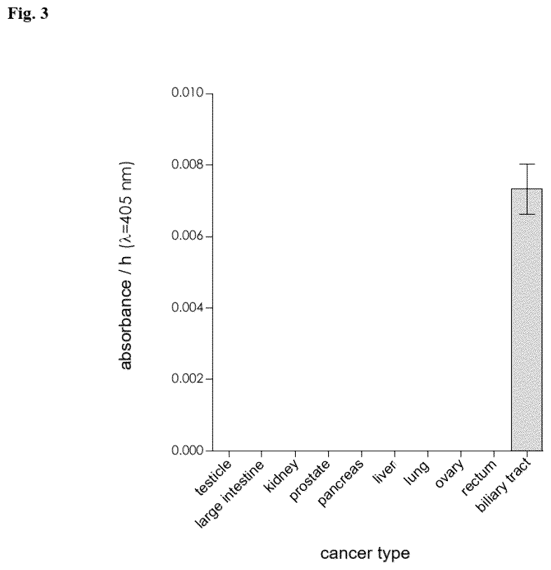

Furthermore, the dependence of the cleavage selectivity of the substrate, i.e. the compound according to the invention, on the cancer type being examined, was studied. The results of the performed tests are presented in FIG. 3 and they indicate that the tested substrate, i.e. ABZ¹-Glu²-Arg³-Arg⁴-Ala⁵-NH₂, incubated with the samples taken from patients with diagnosed following cancers: testicle cancer, large intestine cancer, kidney cancer, prostate cancer, pancreas cancer, liver cancer, lung cancer, ovarian cancer, and rectum cancer, is not subject to cleavage and does not cause an increase in absorbance within the specified range. The samples tested were, in each case, a mixture of 20 samples derived from each of the cancers studied. This indicates cleavage selectivity of the compounds according to the invention, which makes them suitable for the specific detection of enzymatic activity specific to biliary tract cancer and specific diagnosis of biliary tract cancer.

Table 2 below presents the obtained measurements results for each sample in triplicate.

TABLE 2
Results of analysis of selectivity of the cleavage

| | | | |
|---|---|---|---|
| Testicle | 0 | 0 | 0 |
| Large intestine | 0 | 0 | 0 |
| Kidney | 0 | 0 | 0 |
| Prostate | 0 | 0 | 0 |
| Pancreeas | 0 | 0 | 0 |
| Liver | 0 | 0 | 0 |
| Lung | 0 | 0 | 0 |
| Ovary | 0 | 0 | 0 |
| Rectum | 0 | 0 | 0 |
| Biliary tract | 0.0075 | 0.0084 | 0.0071 |

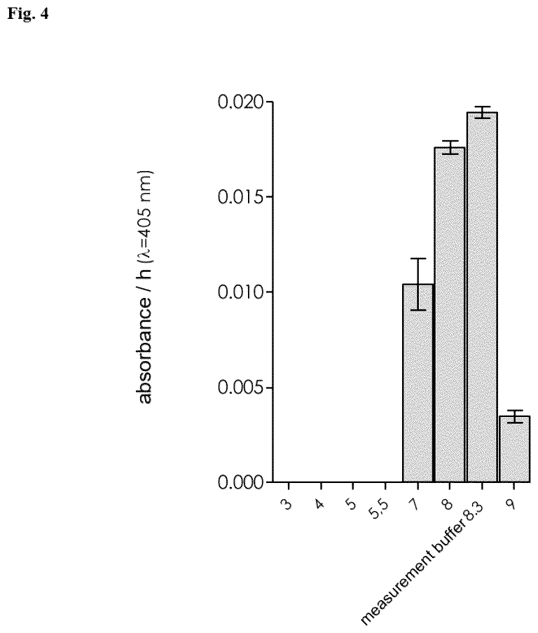

Furthermore, measurements concerning the dependence of the proteolytic activity of the representative compound according to the invention on the reaction pH were performed. The experiment has shown that the studied material has at least one enzyme exhibiting maximum activity at alkaline pH (FIG. 4).

The analyses carried out confirmed suitability of the compounds according to the invention for the sensitive and specific detection of enzymatic activity specific to biliary tract cancer and, by the same, their suitability also for the specific diagnosis of biliary tract cancer, and as a diagnostic marker for biliary tract cancer. The mechanism of action of the compounds according to the invention consists in their specific enzymatic cleavage at the position that leads to the release of free chromophore molecules, which generates a measurable optical signal that can be used for diagnostic purposes, in particular in the diagnosis of biliary tract cancer according to the present invention.

1-29. (canceled)

30. A compound having formula 1: 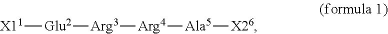
wherein X1 comprises or consists of molecule C1 and X2 comprises or consists of molecule C2,
wherein the pair of molecules C1 and C2 is a pair of a fluorescence donor and a fluorescence acceptor,
and wherein the compound undergoes enzymatic cleavage into the fragments X1-Glu-Arg-Arg-Ala-OH (fragment 1) and X2 (fragment 2) with a generation of a measurable optical signal upon spatial separation of molecules C1 and C2.

31. The compound according to claim 30, which compound undergoes hydrolytic cleavage, preferably proteolytic; and/or the wherein the pair of molecules C1 and C2 is selected from the group consisting of: 2-aminobeznoic acid (ABZ)/5-amino-2-nitrobenzoic acid (ANB), (ABZ)/pNA, ABZ/ANB-NH₂, ABZ/DNP, ABZ/EDDNP, EDANS/DABCYL, TAM/DANSYL, ABZ/Tyr(3-NO₂), preferably the pair of C1 and C2 is ABZ/pNA or ABZ/ANB-NH₂; and/or wherein the compound has a formula of: ABZ-Glu-Arg-Arg-Ala-ANB-NH₂ (formula 2) or a formula of: ABZ-Glu-Arg-Arg-Ala-pNA (formula 3); and/or wherein the hydrolytic cleavage of the compound generates the following fragment 1: ABZ-Glu-Arg-Arg-Ala-OH and fragment 2: ANB-NH₂.

32. An in vitro method for detecting enzymatic activity present in a subject's body fluid deriving from biliary tract cancer cells, comprising:
a) contacting the body fluid sample with the compound having formula 1: 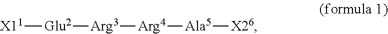
wherein X1 comprises or consists of molecule C1 and X2 comprises or consists of molecule C2,
wherein the pair of molecules C1 and C2 is a pair of a fluorescence donor and a fluorescence acceptor,
and wherein the compound undergoes enzymatic cleavage into the fragments X1-Glu-Arg-Arg-Ala-OH (fragment 1) and X2 (fragment 2), and
b) detecting a measurable optical signal which is generated upon spatial separation of molecules C1 and C2.

33. The in vitro method according to claim 32, wherein the enzymatic activity is hydrolytic activity, preferably proteolytic activity; and/or wherein the compound has a formula of: ABZ-Glu-Arg-Arg-Ala-ANB-NH₂ (formula 2) or a formula of: ABZ-Glu-Arg-Arg-Ala-pNA (formula 3); and/or wherein the body fluid is urine, preferably human urine.

34. An in vitro method for diagnosis of biliary tract cancer, wherein the presence or absence of biliary tract cancer in a subject is detected by measuring enzymatic activity specific to biliary tract cancer in a body fluid sample from the examined subject, and wherein the absence of the said enzymatic activity indicates the absence of biliary tract cancer whereas the presence of the said enzymatic activity indicates the presence of biliary tract cancer, wherein the measurement of the said enzymatic activity is performed using the compound having formula 1: 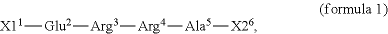
wherein X1 comprises or consists of molecule C1 and X2 comprises or consists of molecule C2,
wherein the pair of molecules C1 and C2 is a pair of fluorescence donor and fluorescence acceptor,
and wherein the said compound undergoes enzymatic cleavage into the fragments X1-Glu-Arg-Arg-Ala-OH (fragment 1) and X2 (fragment 2) with the generation of a measurable optical signal upon spatial separation of molecules C1 and C2.

35. The in vitro method according to claim 34, wherein the detection of enzymatic activity is carried out by
a) contacting the body fluid sample with the compound having formula 1: 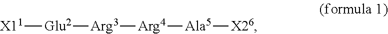
wherein X1 comprises or consists of molecule C1 and X2 comprises or consists of molecule C2,
wherein the pair of molecules C1 and C2 is a pair of a fluorescence donor and a fluorescence acceptor,
and wherein the compound undergoes enzymatic cleavage into the fragments X1-Glu-Arg-Arg-Ala-OH (fragment 1) and X2 (fragment 2), and
b) detecting a measurable optical signal which is generated upon spatial separation of molecules C1 and C2.

36. The in vitro method according to claim 34, wherein the diagnosis of biliary tract cancer comprises the detection of primary biliary tract cancer, detection of Minimal Residual Disease after surgical resection of biliary tract cancer and/or detection of biliary tract cancer recurrence; and/or wherein the body fluid sample is incubated with the said compound in a measurement buffer having neutral or alkaline pH, preferably physiological, within the range of sample-to-measurement buffer ratio of 1:2 to 1:10, preferably 1:5; and/or wherein the compound is used at a concentration of 0.1-10 mg/mL, in particular 0.25-7.5 mg/mL; and/or wherein the compound has a formula of: ABZ-Glu-Arg-Arg-Ala-ANB-NH₂ (formula 2) or formula of: ABZ-Glu-Arg-Arg-Ala-pNA (formula 3); and/or the sample a urine sample, preferably human urine; and/or wherein the measurement of the enzymatic activity comprises the measurement of absorbance intensity in the range of 300-500 nm, preferably 380-430 nm, in particular 405 nm, during 40-60 minutes, at a temperature within the range of 25-40° C., preferably 36-38° C.

37. A kit comprising the compound as defined in claim 30 and a measurement buffer.

38. The kit according to claim 37, wherein the compound is the compound having formula 2: ABZ-Glu-Arg-Arg-Ala-ANB-NH₂ (formula 2) or the compound having formula 3: ABZ-Glu-Arg-Arg-Ala-pNA (formula 3).

39. A method for a treatment of biliary tract cancer, comprising
a) detecting the presence of enzymatic activity specific to biliary tract cancer in a body fluid sample from the examined subject by i) contacting the body fluid sample with the compound having formula 1 as defined in claim 30, ii) detecting a measurable optical signal which is generated upon spatial separation of molecules C1 and C2 following enzymatic cleavage of the compound, and b) applying a treatment of biliary tract cancer in the subject if the presence of the said enzymatic activity is found in the said sample; and,
b) applying a treatment of biliary tract cancer in the subject if the presence of the said enzymatic activity is found in the said sample.

40. The method according to claim 39, wherein after the end of the treatment in accordance with point b), the said enzymatic activity specific to biliary tract cancer is monitored at predetermined time intervals; and/or wherein as the sample a urine sample, preferably human urine, is used; and/or wherein as the compound the compound having formula of: ABZ-Glu-Arg-Arg-Ala-ANB-NH₂ (formula 2) or formula of: ABZ-Glu-Arg-Arg-Ala-pNA (formula 3) is used.
